(19) United States
(12) Patent Application Publication
(10) Pub. No.: US 2010/0227011 A1
(43) Pub. Date: Sep. 9, 2010

(54) REGULATION OF MAMMALIAN KERATINOUS TISSUE USING PERSONAL-CARE COMPOSITIONS COMPRISING A TURMERONE COMPOUND

(76) Inventors: Dennis Eugene Kuhlman, Liberty, OH (US); Rajesh Kant Mishra, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/709,769

(22) Filed: Feb. 22, 2010

Related U.S. Application Data
(60) Provisional application No. 61/154,941, filed on Feb. 24, 2009.

(51) Int. Cl. A61K 36/906; A61K 8/30; A61K 8/36; A61Q 19/02; A61Q 5/08; A61Q 17/04

(52) U.S. Cl. 424/756; 424/60; 424/62

(57) ABSTRACT

Personal-care compositions for improving keratinous tissue conditions, particularly skin lightening, comprising a turmerone compound, a safe and effective amount of at least one additional skin and/or hair care active, and a dermatologically acceptable carrier are provided. Suitable additional skin and/or hair care actives are a sugar amine, a vitamin B₃ compound, a retinoid, a peptide, a hexamidine, a sunscreen, a water-soluble vitamin, an oil-soluble vitamin, an additional skin lightener, and combinations thereof. Methods for regulating the condition of mammalian keratinous tissue and for lighening skin by topically applying the personal-care compositions are also provided.

### CROSS REFERENCE TO RELATED APPLICATION

This application claims the benefit of Provisional Application Ser. No. 61/154,941, filed Feb. 24, 2009.

### TECHNICAL FIELD

The present invention relates to personal-care compositions comprising a turmerone compound and additional skin and/or hair care actives. Such compositions are useful for regulating the condition of mammalian keratinous tissue needing such treatments, particularly skin lightening.

### BACKGROUND OF THE INVENTION

Currently, a number of personal-care products are available to consumers, which are directed toward improving the health and physical appearance of keratinous tissues such as the skin, hair, and nails. The majority of these products are directed to delaying, minimizing or even eliminating skin wrinkling and histological changes typically associated with the aging of skin or environmental damage to human skin. However, there also exists a need for cosmetic agents to prevent, retard, and/or treat uneven skin tone by acting as a lightening or pigmentation reduction cosmetic agent.

### SUMMARY OF THE INVENTION

Applicants have discovered that topical compositions that comprise certain actives may be used to provide prophylactic as well as therapeutic treatments for keratinous tissue conditions, particularly skin-lightening.

One exemplary embodiment of the present invention is directed to a personal-care composition comprising a turmerone compound; a safe and effective amount of at least one additional skin and/or hair care active selected from the group consisting of a sugar amine, a vitamin B₃ compound, a retinoid, a peptide, a hexamidine, a sunscreen, a water-soluble vitamin, an oil-soluble vitamin, an additional skin lightener, and combinations thereof; and a dermatologically acceptable carrier. Optionally, the personal-care composition may comprise phytosterol, dialkanoyl hydroxyproline, salicylic acid, n-acyl amino acid compound, hesperedin, mustard seed extract, glycyrrhizic acid, glycyrrhetinic acid, carnosine, butylated hydroxytoluene and butylated hydroxyanisole, menthyl anthranilate, cetyl pyridinium chloride, ergothioneine, vanillin or its derivatives, diethylhexyl syrinylidene malonate, melanostatine, sterol esters, dehydroacetic acid, idebenone, yeast extract, beta glucans, alpha glucans, and combinations thereof.

Another exemplary embodiment of the present invention is directed to a personal-care composition comprising from about 0.001% to about 10% of a turmerone compound; from about 0.001% to about 20% of a vitamin B₃ compound; from about 0.001% to about 10% of a retinoid; from about 1×10⁻⁶% to about 20% of a peptide; and a dermatologically acceptable carrier. The personal-care composition may further comprise a sunscreen.

Another exemplary embodiment of the present invention is directed to a method for regulating the condition of mammalian keratinous tissue comprising the step of topically applying to the keratinous tissue needing such treatment, a safe and effective amount of a personal-care composition in accordance with the invention. Another exemplary embodiment is directed to a method for lightening skin comprising the step of topically applying the personal-care composition.

### DETAILED DESCRIPTION OF THE INVENTION

The composition of the present invention may be used in skin care, cosmetic, and hair care products, non-limiting uses of which include moisturizers, conditioners, anti-aging compounds, skin-lightening compounds, and combinations thereof. The composition may be applied to keratinous tissue of the face, neck, hands, arms and other areas of the body.

In all embodiments of the present invention, all percentages are by weight of the personal-care composition, unless otherwise specified. All ratios are weight ratios, unless specifically stated otherwise. All numeric ranges are inclusive of narrower ranges. The number of significant digits conveys neither limitation on the indicated amounts nor on the accuracy of the measurements. All measurements are understood to be made at about 25° C. and at ambient conditions, where “ambient conditions” means conditions under about one atmosphere of pressure and at about 50% relative humidity.

“Personal-care composition,” as used herein, means compositions suitable for topical application on mammalian keratinous tissue.

“Keratinous tissue,” as used herein, refers to keratin-containing layers disposed as the outermost protective covering of mammals which includes, but is not limited to, skin, hair, nails, cuticles, etc.

“Dermatologically acceptable,” as used herein, means that the compositions or components described are suitable for use in contact with human keratinous tissue without undue toxicity, incompatibility, instability, allergic response, and the like.

“Safe and effective amount,” as used herein, means an amount sufficient to significantly induce a positive benefit but low enough to avoid serious side effects.

“Regulating keratinous tissue condition,” as used herein, includes prophylactically regulating and/or therapeutically regulating keratinous tissue condition. Regulating keratinous tissue condition may involve one or more of the following benefits: thickening (i.e., building the epidermis and/or dermis layers of the skin and/or the subcutaeous layers such as fat and muscle and where applicable the keratinous layers of the nail and hair shaft) to reduce atrophy (e.g., of the skin); increasing the convolution of the dermal-epidermal border; decreasing non-melanin skin discoloration such as under eye circles, blotching (e.g., uneven red coloration due to, e.g., rosacea) (hereinafter referred to as “red blotchiness”), and sallowness, decreasing discoloration caused by telangiectasia or spider vessels, decreasing discolorations due to melanin (e.g., pigment spots, age spots, uneven pigmentation) and other chromophores in the skin (e.g., lipofuscin, protein crosslinks such as those that occur with glycation, and the like). Regulating skin condition involves improving skin appearance and/or feel. As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like in the skin which may be detected visually or by feel). As used herein, therapeutically regulating skin condition includes ameliorating (e.g., diminishing, minimizing and/or effacing) discontinuities in skin.

“Hyperpigmentation,” as used herein, refers to an area of skin wherein the pigmentation is greater than that of an adjacent area of skin (e.g., a pigment spot, an age spot, and the like).

“Post-inflammatory hyperpigmentation,” as used herein, refers to the changes in melanin content as a response to an inflammatory event (e.g., acne, scratch, insect sting or bite, sunburn, etc), especially in dark skin subjects.

“Sallowness,” as used herein, means the pale color, yellow color or the like condition of skin that occurs as a result of a loss of, damage to, alterations to, and/or abnormalities in skin components such that they become colored (e.g., yellow in color) due to processes such as protein glycation and accumulation of lipofuscin or in the decrease in peripheral blood flow that typically accompanies skin aging.

“Skin lightening,” as used herein, refers to the prevention, retarding, and/or treating of hyperpigmentation, post-inflammatory hyperpigmentation, sallowness, and uneven skin tone. Skin lightening may occur through multiple mechanisms including anti-oxidant mechanisms, trypsin inhibition, anti-inflammatory mechanisms, nitric oxide scavenging, tyrosinase inhibition, etc.

The compositions of the present invention may be used for topical application to regulate keratinous tissue condition. Regulation of keratinous tissue condition is often required due to conditions that may be induced or caused by factors internal and/or external to the body. The compositions of the present invention, including the essential and optional components thereof, are described in detail hereinafter.

### I. COMPONENTS

### A. Turmerone Compound

The present invention comprises a safe and effective amount of a turmerone compound. As used herein, “turmerone compound” means a turmeric (curcuma longa) extract comprising at least 65% of turmerone. The turmerone compound may comprise ar-turmerone, α-turmerone, β-turmerone, and combinations thereof. When topically applied, the turmerone compound acts as a skin lightener, improving skin tone and skin radiance. Oxygen radicals are produced in the skin in response to many stimuli, such as exposure to UV and irritants. Such radicals are also produced as by-products of normal cell or tissue metabolism. Oxygen radicals can stimulate pigment cells (melanocytes) to increase production of melanin. The turmerone compound has anti-oxidant properties and thus can scavenge oxygen radicals before they stimulate the melanocytes. A suitable turmerone compound is supplied under the tradename TEGO® Turmerone by Evonik Degussa GmbH, Essen, Germany. The composition may comprise from about 0.001% to about 10%, from about 0.01% to about 5%, or from about 0.1% to about 1%, by weight of the composition, of a turmerone compound.

### B. Additional Skin and/or Hair Care Actives

The composition of the present invention may comprise one or more skin and/or hair care actives. Suitable actives include, but are not limited to, a sugar amine, a vitamin B₃ compound, a retinoid, a peptide, a hexamidine, a sunscreen, a water-soluble vitamin, an oil-soluble vitamin, an additional skin lightener, and combinations thereof.

1. Sugar Amine

The compositions of the present invention may comprise a sugar amine, also known as amino sugars, and their salts, isomers, tautomers and derivatives. As used herein, “sugar amine” refers to a pure sugar amine compound or a mixture of sugar amine compounds (e.g., extracts from natural sources or mixtures of synthetic materials) of synthetic or natural origin. For example, glucosamine is generally found in many shellfish and can also be derived from fungal sources. Examples of sugar amines useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). In one embodiment, the sugar amine is glucosamine, or D-glucosamine and N-acetyl glucosamine, or N-acetyl-D-glucosamine. Additionally, combinations of two or more sugar amines may be used. The composition may comprise from about 0.01% to about 15%, alternatively from about 0.1% to about 10%, and alternatively from about 0.5% to about 5%, by weight of the composition, of a sugar amine.

2. Vitamin B₃ Compound

The compositions of the present invention may include a safe and effective amount of a vitamin B₃ compound. As used herein, “vitamin B₃ compound” means a compound having the formula:

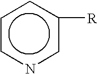

wherein R is —CONH₂ (i.e., niacinamide), —COOH (i.e., nicotinic acid) or —CH₂OH (i.e., nicotinyl alcohol); derivatives thereof; and salts of any of the foregoing. Vitamin B₃ compounds are particularly useful for regulating skin condition as described in U.S. Pat. No. 5,939,082. In one embodiment, the vitamin B₃ compound is niacinamide. The composition may comprise from about 0.001% to about 20%, or from about 0.1% to about 10%, or from about 0.5% to about 7%, by weight of the composition, of a vitamin B₃ compound.

3. Retinoid

The compositions of this invention may comprise a safe and effective amount of a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. In one embodiment, the retinoid is retinyl propionate. The composition may comprise from about 0.001% to about 10%, or from 0.01% to about 1%, or from about 0.01% to about 0.5%, by weight of the composition, of a retinoid.

4. Peptide

The compositions of the present invention may comprise a safe and effective amount of a peptide. As used herein, “peptide” refers to peptides comprising ten or fewer amino acids and their derivatives, isomers, and complexes with other species such as metal ions (e.g., copper, zinc, manganese, magnesium, and the like). In one embodiment, dipeptides, pentapeptides, or combinations thereof are preferred.

In certain embodiments, the peptides contain at least one basic amino acid (e.g., histidine, lysine, arginine). Example peptides are the dipeptide carnosine (beta-ala-his), the tripeptide gly-his-lys, the tripeptide his-gly-gly, the tripeptide gly-gly-his, the tripeptide gly-his-gly, the tetrapeptide gly-gln-pro-arg, the pentapeptide lys-thr-thr-lys-ser, lipophilic derivatives of peptides, and metal complexes of the aforementioned (e.g., copper complex of the tripeptide his-gly-gly (also known as Iamin)). Other suitable peptides include Peptide CK (arg-lys-arg); Peptide CK+ (ac-arg-lys-arg-NH2); and Peptide E, arg-ser-arg-lys. Suitable pentapeptides are disclosed in U.S. Pat. No. 6,492,326.

Examples of useful peptide derivatives include, but are not limited to, peptides derived from palmitoyl-lysine-threonine (pal-KT) and palmitoyl-lysine-threonine-threonine-lysine-serine (pal-KTTKS, available in a composition known as Matrixyl®), palmitoyl-glycine-glutamine-proline-arginine (pal-GQPR, available in a composition known as Rigin®), these three being available from Sederma, and Cu-histidine-glycine-glycine (Cu-HGG, also known as Iamin). Another example is Palestrina®, comprising palmitoyl dipeptide-7, available from Croda Inc. A further example includes carnosine (beta-alanine-histidine). In one embodiment, the peptide is ProMatrixyl® which contains palmitoyl pentapeptide-4 and is commercially available from Sederma. The composition may comprise from about 1×10⁻⁶% to about 20%, or from about 1×10⁻⁵% to about 10%, or from about 1×10⁻⁴% to about 5%, by weight of the composition, of a peptide.

5. Hexamidine

The compositions of the present invention may comprise a safe and effective amount of a hexamidine. As used herein, “hexamidine” means a compound having the formula:

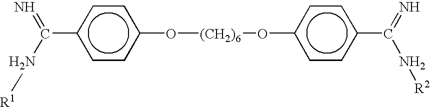

wherein R¹ and R² are optional or are organic acids (e.g., sulfonic acids, etc.), and its salts, isomers, tautomers, and derivatives. In one embodiment, the hexamidine is hexamidine diisethionate, available under the tradename Elastab® HP100 from Laboratoires Serobiologiques (Pulnoy, France). The composition may comprise from about 0.0001-25%, alternatively from about 0.001-10%, and alternatively from about 0.01-5%, by weight of the composition, of a hexamidine.

6. Sunscreen

The compositions of the present invention may comprise a safe and effective amount of a sunscreen. As used herein, “sunscreen” includes both sunscreen agents and physical sunblocks. Sagarin, et al., at Chapter VIII, pages 189 et seq., of Cosmetics Science and Technology (1972), discloses numerous suitable sunscreens. Particularly suitable sunscreens are 4,4′-t-butyl methoxydibenzoyl-methane, 2-ethylhexyl-p-methoxycinnamate, 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, octisalate, zinc oxide, titanium dioxide, and mixtures of these compounds. In one embodiment, the composition comprises more than one sunscreen. The composition may comprise from about 1% to about 25%, or from about 2% to about 10%, by weight of the composition, of a sunscreen.

7. Water-Soluble Vitamin

The compositions of the present invention may comprise a safe and effective amount of a water-soluble vitamin. Examples of water-soluble vitamins include, but are not limited to, water-soluble versions of vitamin B (such as vitamin B₅ and vitamin B₆ (e.g., pyridoxine)), vitamin B derivatives, vitamin C (such as ascorbyl glucoside), vitamin C derivatives (such as magnesium ascorbyl phosphate, sodium ascorbyl phosphate, and ascorbyl palmitate), vitamin K, vitamin K derivatives, pro-vitamins thereof, and mixtures thereof. In one embodiment, the water-soluble vitamin is panthenol. The composition may comprise from about 0.001% to about 20%, or from about 0.01% to about 10%, or from about 0.1% to about 5%, by weight of the composition, of a water-soluble vitamin.

8. Oil-Soluble Vitamin

The compositions of the present invention may comprise a safe and effective amount of an oil-soluble vitamin. Examples of oil-soluble vitamins include, but are not limited to, oil-soluble versions of vitamin D, vitamin D derivatives, vitamin E, vitamin E derivatives, pro-vitamins thereof, and mixtures thereof. In one embodiment, the oil-soluble vitamin is tocopheryl acetate. The composition may comprise from about 0.001% to about 20%, or from about 0.01% to about 10%, or from about 0.1% to about 5%, by weight of the composition, of an oil-soluble vitamin.

9. Additional Skin Lightener

The personal-care composition may comprise a safe and effective amount of a skin lightener in addition to a turmerone compound. Suitable additional skin lighteners include those known in the art, including kojic acid, hydroquinone, arbutin, tranexamic acid, N-undecylenoyl-L-phenylalanine, pyridoxime, ascorbic acid and derivatives thereof (e.g., magnesium ascorbyl phosphate or sodium ascorbyl phosphate, ascorbyl glucoside, and the like). Other skin lighteners suitable for use herein include Acitwhite (COGNIS), Emblica (RONA), Azeloglicina (SINERGA), and extracts (e.g., mulberry extract). The composition may comprise from about 0.1% to about 10%, alternatively from about 0.2% to about 5%, alternatively from about 0.5% to about 2%, by weight of the composition, of an additional skin lightener.

### C. Dermatologically Acceptable Carrier

The personal-care compositions of the present invention also comprise a dermatologically acceptable carrier for the active materials. The phrase “dermatologically acceptable carrier,” as used herein, means that the carrier is suitable for topical application to the keratinous tissue, has good aesthetic properties, is compatible with the actives of the present invention and any other components, and will not cause any safety or toxicity concerns. The composition may comprise the dermatologically acceptable carrier in ranges from about 10% to about 99.99%, or from about 60% to about 99.9%, or from about 70% to about 98%, or from about 80% to about 95%, by weight of the composition.

The carrier may be in a wide variety of forms. Suitable carriers may include an emulsion such as oil-in-water emulsions and water-in-oil emulsions, e.g., silicone-in-water or water-in-silicone emulsions. Other emulsion carriers may include water-in-oil-in-water and oil-in-water-in-silicone emulsions. As will be understood by the skilled artisan, a given component will distribute primarily into either the water or oil phase, depending on the water solubility/dispensability of the component in the composition.

Emulsions according to the present invention generally comprise a solution as described above and a lipid and/or oil. Lipids and oils may be derived from animals, plants, or petroleum and may be natural or synthetic (i.e., man-made). Emulsions may also comprise a humectant, such as glycerin. The composition may comprise from about 0.1% to about 20%, or from about 1% to about 15%, by weight of the composition, of a humectant.

The composition of the present invention may comprise an emulsifier that may be linear, branched, and/or cross-linked. The composition may comprise from about 0.05% to about 20%, or from about 0.1% to about 10%, or from about 0.2% to about 5%, by weight of the composition, of an emulsifier. Emulsifiers may be nonionic, anionic or cationic. Non-limiting examples of emulsifiers are disclosed in U.S. Pat. No. 3,755,560, U.S. Pat. No. 4,421,769, and McCutcheon's Detergents and Emulsifiers, North American Edition and International Edition, pages 235-246 (1993). Suitable emulsions may have a wide range of viscosities depending on the desired product form.

The compositions of the present invention may also comprise other dermatologically acceptable topical carriers and can also comprise oral carriers. For example, another topical carrier can be a surfactant-containing cleanser (e.g., bar, shampoo, foaming cleanser, liquid cleanser, body wash, cleansing cloth, and the like). In such a carrier, the surfactant can be anionic, cationic, zwitterionic, nonionic, or mixtures of these. Another topical carrier example is a color cosmetic (lipstick, rouge, eye liner, mascara, foundation, nail polish, and the like). An oral carrier can be a beverage, food item, pill, capsule, powder, caplet, and the like.

### D. Optional Components

The personal-care compositions of the present invention may comprise a variety of other dermatologically acceptable components. The CTFA Cosmetic Ingredient Handbook, Second Edition (1992) describes a wide variety of nonlimiting cosmetic and pharmaceutical components commonly used in the skin-care industry, which are suitable for use in the compositions of the present invention. Examples of these component classes include: abrasives; absorbents; aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate); anti-acne agents; anti-atrophy actives; anti-caking agents; anti-cellulite agents; anti-foaming agents; anti-fungal actives; anti-inflammatory agents; anti-microbial agents (e.g., iodopropyl butylcarbamate); anti-oxidants; binders; biological additives; buffering agents; bulking agents; chelating agents; chemical additives; colorants; cosmetic biocides; denaturants; desquamatory actives; drug astringents; external analgesics; film formers or materials, e.g., polymers, for aiding the film-forming properties and substantivity of the composition (e.g., copolymer of eicosene and vinyl pyrrolidone); flavonoids; hair growth regulators; minerals; non-vitamin antioxidants and radical scavengers; oil control agents; opacifying agents; particulate material; pH adjusters; phytosterols and/or plant hormones; propellants; protease inhibitors; reducing agents; sequestrants; skin-conditioning agents; skin soothing and/or healing agents and derivatives; skin treating agents; tanning actives; thickeners; topical anesthetics; tyrosinase inhibitors; vitamins; wrinkle repair actives; and combinations and derivatives thereof. Suitable optional components useful in the present invention include, but are not limited to, phytosterol, dialkanoyl hydroxyproline, salicylic acid, n-acyl amino acid compounds, hesperedin, mustard seed extract, glycyrrhizic acid, glycyrrhetinic acid, carnosine, butylated hydroxytoluene and butylated hydroxyanisole, menthyl anthranilate, cetyl pyridinium chloride, ergothioneine, vanillin or its derivatives, diethylhexyl syrinylidene malonate, melanostatine, sterol esters, dehydroacetic acid, idebenone, yeast extract, beta glucans, alpha glucans, their salts, their derivatives, their precursors, and combinations thereof. These actives and others are provided in further detail in U.S. Application Publication Nos. US2006/0275237A1, US2004/0175347A1, and US2006/0263309A1. The personal-care composition may comprise from about 0.001% to about 10%, by weight, of optional components.

### IV. METHODS FOR REGULATING KERATINOUS TISSUE CONDITION

In one embodiment, the composition of the present invention may be applied topically to keratinous tissue to regulate keratinous tissue condition. Regulating keratinous tissue condition involves topically applying to the keratinous tissue a safe and effective amount of a composition of the present invention. In another embodiment, the composition of the present invention may be applied topically to keratinous tissue to lighten skin. The amount of the composition that is applied, the frequency of application, and the period of use will vary widely depending upon the level of skin and/or hair care actives and/or other components of a given composition and the level of regulation desired.

### VI. EXAMPLES

The following describe non-limiting examples of the composition. The reported percentages indicate the weight of the component expressed as a percentage of the total weight of the personal-care composition. Each Example may comprise one or more of the optional ingredients in amounts also disclosed herein. The examples are given solely for the purpose of illustration and are not to be construed as limitations of the present invention, as many variations thereof are possible without departing from the spirit and scope of the invention, which would be recognized by one of ordinary skill in the art.

The Examples are generally prepared by conventional methods such as are known in the art of making topical compositions. Such methods typically involve mixing of the ingredients in one or more steps to a relatively uniform state, with or without heating, cooling, application of vacuum, and the like. The Examples are preferably prepared such as to optimize stability (physical stability, chemical stability, photostability) and/or delivery of the active materials.

| | 1 | 2 | 3 | 4 | 5 |
|---|---|---|---|---|---|
| Components | (wt %) | (wt %) | (wt %) | (wt %) | (wt %) |
| Allantoin | — | 0.1000 | — | 0.1000 | 0.1000 |
| Argireline¹ | — | 2.0000 | — | — | — |
| Benzyl Alcohol | — | 0.2500 | 0.5000 | 0.2500 | 0.2500 |
| Borage Seed Oil | — | — | — | 1.0000 | 1.0000 |
| Brij 72² | 0.2500 | — | — | — | — |
| Brij 721³ | 0.5000 | — | — | — | — |
| Caffeine | — | 1.0000 | — | — | — |
| Cetyl Alcohol/CO1695F | 1.3300 | 0.6400 | 0.6400 | 0.5670 | 0.5670 |
| Citric Acid | 0.0700 | - | 0.1000 | 0.0100 | 0.0100 |
| DC 1503 Fluid⁴ | — | 2.0000 | 2.0000 | 2.0000 | 2.0000 |
| Disodium EDTA | 0.1000 | 0.1000 | 0.1000 | 0.1000 | 0.1000 |
| Emulgade 68/50⁵ | — | 0.4000 | 0.4000 | 0.2000 | 0.2000 |
| Evening Primrose Oil | — | — | — | 1.0000 | 1.0000 |
| Glycacil L⁶ | — | — | 0.0900 | — | — |
| Glycerin | 3.0000 | 12.0000 | 10.0000 | 12.0000 | 12.0000 |
| Glydant Plus Liquid⁷ | | 0.300000 | | 0.3000 | 0.3000 |
| Hydrobrite 1000PO⁸ | 1.7500 | — | — | — | — |
| Hydrolite-5⁹ | — | — | 1.0000 | — | — |
| Kathon CG¹⁰ | 0.0300 | — | — | — | — |
| Lanette 22¹¹ | | 0.8000 | 0.8000 | 0.5000 | 0.5000 |
| Lonzaine 16SP¹² | 4.4390 | — | — | — | — |
| Microthene FN-510-00¹³ | — | — | 2.0000 | 2.0000 | 2.0000 |
| Myrj 59¹⁴ | — | 0.2000 | 0.2000 | 0.1000 | 0.1000 |
| NatureBead R20¹⁵ | 0.5000 | — | — | — | — |
| Neo Heliopan OS¹⁶ | — | — | 4.0000 | — | — |
| Neolone 950¹⁷ | — | — | 0.0500 | — | — |
| Niacinamide | 0.5000 | 6.000000 | 6.0000 | 6.0000 | 6.0000 |
| Panthenol | 0.1000 | 1.0000 | 1.0000 | 1.0000 | 1.0000 |
| Parsol 1789¹⁸ | — | — | 2.0000 | — | — |
| Parsol 340¹⁹ | — | — | 1.2500 | — | — |
| Parsol HS²⁰ | — | — | 1.0000 | — | — |
| Permethyl 101A²¹ | — | 3.0000 | — | 3.0000 | 3.0000 |
| Plantaren 2000 UP²² | 2.0000 | — | — | — | — |
| PM1P75CSI or PM1P75KSI²³ | — | 0.600000 | — | 0.600000 | 0.600000 |
| Prodew 400²⁴ | — | 0.1000 | — | — | — |
| ProMatrixyl²⁵ | 0.1000 | 0.4710 | 0.4710 | 0.706000 | 0.706000 |
| Propyltex 50pc-Powder/Bead²⁶ | 0.5000 | — | — | — | — |
| Retinyl Propionate | 0.1000 | 0.1000 | 0.1000 | 0.1000 | 0.1000 |
| Schercemol 318²⁷ | — | 1.3300 | 1.3300 | 1.3300 | 1.3300 |
| Sepigel 305²⁸ | — | 2.250000 | 2.250000 | 2.250000 | 2.250000 |
| Sepitonic M3²⁹ | — | — | — | 0.5000 | 0.5000 |
| Shebu Refined³⁰ | — | — | — | 2.0000 | 2.0000 |
| Skin Lightener³¹ | — | — | — | — | 1.0000 |
| Sodium Hyaluronate | — | 0.0500 | 0.0100 | 0.0100 | 0.0100 |
| Stearyl Alcohol | 1.3300 | 0.9600 | 0.9600 | 0.7330 | 0.7330 |
| Stepanol WAC³² | 2.3780 | — | — | — | — |
| Super White Protopet/Petrolatum³³ | 3.2500 | 0.5000 | — | — | — |
| TegoTurmerone³⁴ | 0.1000 | 0.5000 | 0.5000 | 0.5000 | 0.5000 |
| Timiron Splendid Gold³⁵ | — | 0.5000 | — | — | — |
| Tocopheryl Acetate | 0.1000 | 0.5000 | 0.5000 | 0.5000 | 0.5000 |
| Tospearl 2000³⁶ | — | 0.5000 | — | 0.5000 | 0.5000 |
| Triethanolamine | — | — | 0.7500 | — | — |
| Varisoft TA-100³⁷ | 1.0000 | — | — | — | — |
| Water | qs | qs | qs | qs | qs |
| Wickenol 111³⁸ | 15.0000 | — | — | — | — |
| TOTAL | 100 | 100 | 100 | 100 | 100 |

¹Water, Phenoxyethanol, Methylparaben, Ethylparaben, Butylparaben, Propylparaben, Isobutylparaben, and Acetyl Hexapeptide-8 from Lipotec
²Steareth-2 from Uniqeme.
³Steareth-21 from Uniqeme.
⁴Dimethicone and Dimethiconal from Dow Corning.
⁵Cetearyl Glucoside and Cetearyl Alcohol from Cognis.
⁶Iodopropynyl Butylcarbamate, PEG-4 Laurate, PEG-4 Dilaurate, and PEG-4 from Lonza.
⁷DMDM Hydantoin, Iodopropynyl butylcarbamate, Butylene glycol, and Water from Lonza.
⁸Mineral Oil from Witco/Crompton/Sonneborn.
⁹Pentylene Glycol from Symrise.
¹⁰Methylchloroisothiazolinone, Methylisothiazolinone, Magnesium Chloride, Magnesium Nitrate, and Water from Rohm & Haas.
¹¹Behenyl Alcohol from Cognis.
¹²Water, Sodium Chloride, Ethanol, Sodium Glycolate, and Cetyl Betaine from Lonza.
¹³Polyethylene from Equistar.
¹⁴PEG-100 Stearate from Uniqema.
¹⁵Oryza Sativa (Rice) Bran Wax from Symrise.
¹⁶Octisalate from Symrise.
¹⁷Methylisothiazolinone and Water from Rhom & Haas.
¹⁸Avobenzone from DSM.
¹⁹Octocrylene from DSM.
²⁰Ensulizole from DSM.
²¹Isohexadecane from Presperse/Uniqema.
²²Decyl Glucoside and Water from Cognis.
²³Titanium Dioxide, Isohexadecane, Polyhydroxystearic Acid, and Triethoxycaprylylsilane from KOBO.
²⁴Water, Sodium PCA, Betaine, Sorbitol, Glycine, Alanine, Proline, Serine, Threonine, Arginine, Lysine, Glutamic Acid, Methylparaben, and Propylparaben from Ajinomoto.
²⁵Glycerin, Water, PEG-100 Stearate, Benzyl Alcohol, and Palmitoyl Pentapeptide-4 from Sederma.
²⁶Polypropylene from Micropowder/Presperse.
²⁷Isopropyl Isostearate from Noveon.
²⁸Water, Polyacrylamide, C13-14 Isoparaffin, and Laureth-7 from Seppic.
²⁹Water, Zinc Gluconate, Magnesium Aspartate, Phenoxyethanol, and Copper Gluconate from Seppic.
³⁰Butyrospermum Parkii from Rita.
³¹E.g., kojic acid, hydroquinone, arbutin, tranexamic acid, N-undecylenoyl-L-phenylalanine, pyridoxime, ascorbic acid, Acitwhite (COGNIS), Emblica (RONA), Azeloglicina (SINERGA), and extracts.
³²Water and Sodium Lauryl Sulfate from Stepan.
³³Petrolatum from Witco/Crompton.
³⁴Curcuma Longa (Turmeric) Root Extract from Evonik.
³⁵Titanium Dioxide, Mica, and Silica from EMD Chemicals.
³⁶Polymethylsilsesquioxane from GE Silicones/Momentive.
³⁷Distearyldimonium Chloride from Degussa/Evonik.
³⁸Isopropyl Palmitate from Alzo.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal-care composition comprising:
a) a turmerone compound; and
b) a safe and effective amount of at least one additional skin and/or hair care active selected from the group consisting of a sugar amine, a vitamin B₃ compound, a retinoid, a peptide, a hexamidine, a sunscreen, a water-soluble vitamin, an oil-soluble vitamin, an additional skin lightener, and combinations thereof; and
c) a dermatologically acceptable carrier.

2. The personal-care composition of claim 1, comprising from about 0.001% to about 10% of a turmerone compound.

3. The personal-care composition of claim 1, wherein the at least one additional skin and/or hair care active is a vitamin B₃ compound.

4. The personal-care composition of claim 3, wherein the vitamin B₃ compound is niacinamide.

5. The personal-care composition of claim 1, wherein the at least one additional skin and/or hair care active is a retinoid.

6. The personal-care composition of claim 5, wherein the retinoid is retinyl propionate.

7. The personal-care composition of claim 1, wherein the at least one additional skin and/or hair care active is a peptide.

8. The personal-care composition of claim 7, wherein the peptide is selected from the group consisting of dipeptides, pentapeptides, and combinations thereof.

9. The personal-care composition of claim 7, wherein the peptide is palmitoyl pentapeptide-4.

10. The personal-care composition of claim 1, wherein the at least one additional skin and/or hair care active is a hexamidine.

11. The personal-care composition of claim 1, wherein the at least one additional skin and/or hair care active is a sunscreen.

12. The personal-care composition of claim 11, wherein the sunscreen is selected from the group consisting of octisalate, octocrylene, 4,4′-t-butyl methoxydibenzoyl-methane, 2-phenylbenzimidazole-5-sulfonic acid, and combinations thereof.

13. The personal-care composition of claim 1, wherein the at least one additional skin and/or hair care active is a water-soluble vitamin.

14. The personal-care composition of claim 13, wherein the water-soluble vitamin is panthenol.

15. The personal-care composition of claim 1, wherein the at least one additional skin and/or hair care active is an oil-soluble vitamin.

16. The personal-care composition of claim 15, wherein the oil-soluble vitamin is tocopheryl acetate.

17. The personal-care composition of claim 1, wherein the at least one additional skin and/or hair care active is an additional skin lightener.

18. The personal-care composition of claim 17, wherein the additional skin lightener is selected from the group consisting of kojic acid, hydroquinone, arbutin, tranexamic acid, N-undecylenoyl-L-phenylalanine, pyridoxime, ascorbic acid and derivatives thereof, extracts, and combinations thereof.

19. The personal-care composition of claim 1, further comprising at least one component selected from the group consisting of phytosterol, dialkanoyl hydroxyproline, salicylic acid, n-acyl amino acid compounds, hesperedin, mustard seed extract, glycyrrhizic acid, glycyrrhetinic acid, carnosine, butylated hydroxytoluene and butylated hydroxyanisole, menthyl anthranilate, cetyl pyridinium chloride, ergothioneine, vanillin or its derivatives, diethylhexyl syrinylidene malonate, melanostatine, sterol esters, idebenone, dehydroacetic acid, yeast extract, beta glucans, alpha glucans, and combinations thereof.

20. The personal-care composition of claim 1, further comprising at least one component selected from the group consisting of abrasives, absorbents, aesthetic components, anti-acne agents, anti-atrophy actives, anti-caking agents, anti-cellulite agents, anti-foaming agents, anti-fungal actives, anti-inflammatory agents, anti-microbial agents, anti-oxidants, binders, biological additives, buffering agents, bulking agents, chelating agents, chemical additives, colorants, cosmetic biocides, denaturants, desquamatory actives, drug astringents, external analgesics, film formers, flavonoids, hair growth regulators, minerals, non-vitamin antioxidants and radical scavengers, oil control agents, opacifying agents, particulate material, pH adjusters, phytosterols and/or plant hormones, propellants, protease inhibitors, reducing agents, sequestrants, skin-conditioning agents, skin soothing and/or healing agents and derivatives, skin treating agents, tanning actives, thickeners, topical anesthetics, tyrosinase inhibitors, vitamins, wrinkle repair actives, and combinations thereof.

21. A method of regulating keratinous tissue condition, the method comprising the step of topically applying to keratinous tissue in need of such treatment the composition of claim 1.

22. A method of skin lightening, the method comprising the step of topically applying to keratinous tissue in need of such treatment the composition of claim 1.

23. A personal-care composition comprising:
a. from about 0.001% to about 10% of a turmerone compound;
b. from about 0.001% to about 20% of a vitamin B₃ compound;
c. from about 0.001% to about 10% of a retinoid;
d. from about 1×10⁻⁶% to about 20% of a peptide; and
e. a dermatologically acceptable carrier.
